(19) United States
(12) Patent Application Publication
(10) Pub. No.: US 2013/0266530 A1
(43) Pub. Date: Oct. 10, 2013

(54) METHOD OF COLOURING HAIR FIBRES

(71) Applicants: Ranjit Kaur Bhogal, Sharnbrook (GB); John Casey, Sharnbrook (GB); Shovan Ganguli, Bangalore (IN); Karl John Hunter, Sharnbrook (GB); Jean Hypolites Koek, AT Vlaardingen (NL); Sally Pamela Redfern, Sharnbrook (GB)

(72) Inventors: Ranjit Kaur Bhogal, Sharnbrook (GB); John Casey, Sharnbrook (GB); Shovan Ganguli, Bangalore (IN); Karl John Hunter, Sharnbrook (GB); Jean Hypolites Koek, AT Vlaardingen (NL); Sally Pamela Redfern, Sharnbrook (GB)

(21) Appl. No.: 13/992,326

(22) PCT Filed: Dec. 12, 2011
(86) PCT No.: PCT/EP11/72433

§ 371 Date: Jun. 7, 2013

(30) Foreign Application Priority Data
Dec. 20, 2010 (EP) EP10195893

(51) Int. Cl. A61K 8/49; A61K 8/22; A61K 8/66; A61Q 5/10

(52) U.S. Cl. CPC A61K 8/498; A61Q 5/10; A61K 8/22; A61K 8/66
USPC 424/70.1

(57) ABSTRACT

Hair colourant formulations fall into three main categories designated permanent, semi-permanent and temporary. Permanent hair colourant formulations are oxidative dye systems and generally contain paraphenylene diamine (PPD) and resorcinol, both of which have been shown to cause sensitisation and mutagenicity. Furthermore, severe oxidising conditions are required which in themselves cause skin irritation and sensitization as well as hair fibre damage. The inventive method addresses the aforementioned disadvantages by providing a method of colouring hair fibres, the method comprising the step of applying a hair colour composition, the hair colour composition comprising: (a) (+)-Catechin, (−)-catechin, (+)-epicatechin, (−)-epicatechin or mixtures thereof; (b) A hydrogen peroxide generator or hydrogen peroxide; and (c) A peroxidase and; wherein the composition has a pH of 4.5 to 7.0, preferably less than or equal to 6.0.

A method of colouring hair fibres is disclosed, the method comprising the step of applying a hair colour composition based on (+)-catechin, (−)-catechin, (+)-epicatechin, (−)-epicatechin or mixtures thereof and an enzyme, in particular a peroxidase. The hair colour composition also includes a hydrogen peroxide generator or hydrogen peroxide per se. Optionally the hair colour composition includes metal ions, for example iron or copper, which are able to generate a range of colours for dyeing hair through coordination chemistry with (+)-catechin, (−)-catechin, (+)-epicatechin, (−)-epicatechin or mixtures thereof, or reaction products thereof with the enzyme.

Hair colourant formulations fall into three main categories designated permanent, semi-permanent and temporary. They vary in their degree of wash-fastness where permanent hair colourant formulations last 30-40 shampoo cycles (4-6 weeks) and temporary systems are rinsed out during the first wash. Permanent hair colourant formulations are oxidative dye systems and generally contain paraphenylene diamine (PPD) and resorcinol, both of which have been shown to cause sensitisation and mutagenicity. Furthermore the severe oxidising conditions required (hydrogen peroxide concentrations of 3% or more and a pH of 8.5 or higher for time periods of 20 minutes or more) in themselves can cause skin irritation and sensitization for some individuals as well as hair fibre damage. In addition, permanent hair colourant formulations also contain ammonia, used to swell the hair and lift the cuticle scales to allow penetration of dye precursors, which gives off a strong and unpleasant odour.

Takada et al. (Journal of Oleo Science, 52, 10, 557-563 (2003)) discloses a hair colouring composition comprising laccase and catechin at pH 5.

U.S. Pat. No. 6,572,843 (Novozymes A/S) discloses a method for treating hair combining permanent dyeing and straightening of hair. After application of a relaxer to reduce the covalent disulphide linkages in the keratinous fibres of the hair thereby rendering the hair deformable, a Coprinus sp. peroxidase, hydrogen peroxide and a mediator solution consisting of paraphenylene diamine, 3-aminophenol and 4-aminophenol at pH 5.5 is applied to thereby colour the fibres and simultaneously neutralise the relaxer and fix the hair (by re-establishing the network of crosslinks in the keratinous fibres). U.S. Pat. No. 3,957,424 (The Proctor & Gamble Company) discloses an oxidative colouring process for hair based on hydrogen peroxide and soybean peroxidase which can be carried out under mild oxidising conditions (0.01-1.0% by weight hydrogen peroxide and pH 4 to 10) not requiring a dual oxidation dye precursor comprising an aromatic polyhydric compound and an aromatic amine. In fact either can be used alone as the oxidation dye precursor. What is claimed is a process for colouring hair comprising contacting hair with an effective amount of a solution containing from about 0.01 to about 500 ppm (0.01 to 500 μg/mL) soybean peroxidase, from about 0.01 to about 1.0% by weight hydrogen peroxide, and from about 0.001 to about 6% by weight of an aromatic oxidation dye precursor, and having a pH of from about 4 to 10. In particular, a hair dyeing kit is disclosed comprising a 4 ounce bottle of 1% by weight hydrogen peroxide, 2 g of carboxymethyl cellulose, 20 g starch, 0.07 g soybean peroxidase, 3 g pH 7 buffer and 4 g paraphenylene diamine or equivalent amounts of 4-hydroxy-3-methoxycinnamic acid (ferulic acid) or 2-methoxy-4-(1-propenyl)phenol (isoeugenol). Both latter compounds come under the broad term of phenylpropanoids as they are derived from phenylalanine and the phenylpropanoid pathway, ferulic acid being more specifically a phenylpropenoid and isoeugenol being more specifically a phenylpropene.

The inventive method addresses the aforementioned disadvantages by providing a method of colouring hair fibres which avoids the issues of safety and toxicity associated with the known synthetic oxidative systems, causes less damage by avoiding the use of hydrogen peroxide per se, whilst maintaining colour fastness.

### SUMMARY OF THE INVENTION

In a first aspect of the invention, a method of colouring hair fibres is provided, the method comprising the step of applying a hair colour composition, the hair colour composition comprising:

(a) (+)-Catechin, (−)-catechin, (+)-epicatechin, (−)-epicatechin or mixtures thereof;
(b) A hydrogen peroxide generator or hydrogen peroxide; and
(c) A peroxidase and;
wherein the composition has a pH of 4.5 to 7.0, preferably less than or equal to 6.0.

As consumers will benefit from avoiding the issues around allergic skin reactions, safety and toxicity of current permanent hair colourant formulations, the hair colour composition may be applied more frequently, for example daily, in the form of, for example, a shampoo or conditioner thereby addressing the problem of grey hair or natural hair colour re-growth observed during the intervals between hair dyeing treatments. Furthermore by multiple consecutive applications of the hair colour composition, the consumer can also control the degree of colour desired.

The hair colour composition may comprise 0.01-10, preferably 0.1-5% w/w (+)-catechin, (−)-catechin, (+)-epicatechin, (−)-epicatechin or mixtures thereof.

The peroxidase is selected from those which are suited to neutral or slightly acid pH, that is to say a pH of 4.5 to 7.0, preferably a pH of less than or equal to 6.0. The peroxidase is preferably a non-animal haem peroxidase from class II (fungi) or class III (plants and algae), most preferably obtained from the group consisting of Arabidopsis thaliana, horse radish, barley, peanut soybean, tobacco, and turnip (plants), Chlorophyta spirogyra (green algae), Arthromyces ramosus and Corprinus cinereus (fungi). The hair colour composition may comprise 0.0001-5, preferably 0.001-1% w/w peroxidase.

A hydrogen peroxide generator is preferred because it reduces the aforementioned problems associated with the use of large amounts of hydrogen peroxide whilst providing a constant supply of hydrogen peroxide. Preferably the hydrogen peroxide generator comprises a hydrogen peroxide generating oxidase, a substrate and oxygen. Alternatively a chemical system can be used to generate hydrogen peroxide such as one based on ascorbate and a transition metal ion, or anthraquinone monosulphate and glucose (T. Vuorinen, Carbohydrate Research, 127, 2, 319-325 (15 Apr. 1984)).

The hydrogen peroxide generating oxidase may be selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, α-hydroxy acids, D-glucose, oxalic acid, amino acid and L-galactono-1,4-lactone.

More preferably the hydrogen peroxide generator is selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, amino acid oxidase with amino acid and L-galactono-1,4-lactone with L-galactonolactone oxidase, all in the presence of oxygen.

The hair colour composition may comprise 0.0001-3 preferably 0.001-1, most preferably 0.01-1% w/w hydrogen peroxide. However if a hydrogen peroxide generator is selected, then the hair colour composition may comprise 0.0001-5, preferably 0.001-1% w/w hydrogen peroxide generating oxidase, and 0.01-10, preferably 0.1-5% w/w substrate.

The method of the first aspect may additionally comprise a metal ion suitable for coordinating to (+)-catechin, (−)-catechin, (+)-epicatechin, (−)-epicatechin or mixtures thereof, or the product of the reaction of (+)-catechin, (−)-catechin, (+)-epicatechin, (−)-epicatechin or mixtures thereof in the presence of the peroxidase and hydrogen peroxide. The metal ion may be selected from the group consisting of iron (II), iron (III), copper (I), copper (II), and aluminium (III). The hair colour composition may comprise 0.0001-2, preferably 0.001-0.1% w/w metal ion.

### BRIEF DESCRIPTION OF THE FIGURES

The invention will now be illustrated with reference to the figures which show in:

FIG. 1 the ΔE (FIG. 1a) and L* (FIG. 1b), a* (FIG. 1c) and b* (FIG. 1d) respectively for each of the reactions described in example 1 on the basis of six repeat measurements for each hair switch;

FIG. 2 the ΔE (FIG. 2a) and L* (FIG. 2b), a* (FIG. 2c) and b* (FIG. 2d) respectively for cumulative colour uptake described in example 2 on the basis of five repeat measurements for each hair switch; and

FIG. 3 the ΔE (FIG. 3a) and L* (FIG. 3b), a* (FIG. 3c) and b* (FIG. 3d) respectively for cumulative colour uptake described in example 3 on the basis of five repeat measurements for each hair switch wherein the ΔE values take the ×5 cumulative colour uptake as the baseline and “0” represents the L*, a* and b* values after ×5 cumulative colour uptake.

### DETAILED DESCRIPTION OF THE INVENTION

### Example 1

### A Comparison Between Peroxidase and Laccase

Materials:

Myceliphthora thermophila laccase (51003) was obtained from Novozymes A/S (Denmark)

Horse radish peroxidase (HRP) was obtained from Sigma (UK)

3% aqueous hydrogen peroxide (H₂O₂) was obtained from Sigma (UK)

(+)-Catechin hydrate was obtained from Sigma (UK)

Natural White hair switches were obtained from International Hair Importers (New York, USA)

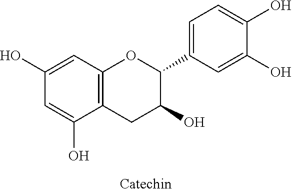 [SMILES: Oc1cc(O)c2c(c1)O[C@H](c1ccc(O)c(O)c1)[C@@H](O)C2]

Method:

Replicate baseline L*a*b* values of the hair switches (−0.5 g) were determined using a Minolta model CM508d spectrophotometer. The hair switches were then incubated in reactions set up as follows (final concentrations in brackets) at 30 degrees centigrade for 15 minutes:

Laccase reaction:

| | |
|---|---|
| 300 μl | 100 mg/ml (+)-catechin hydrate (5 mg/ml) |
| 60 μl | 1000 U/ml laccase (10 U/ml) |
| 5640 μl | 50 mM succinate buffer pH 5.0 |

Horse radish peroxidase/hydrogen peroxide reaction:

| | |
|---|---|
| 300 μl | 100 mg/ml (+)-catechin hydrate (5 mg/ml) |
| 120 μl | 500 U/ml horse radish peroxidase (10 U/ml) |
| 600 μl | 3% aqueous hydrogen peroxide (0.3%) |
| 4980 μl | 50 mM succinate buffer pH 5.0 |

(+)-Catechin control

| | |
|---|---|
| 300 μl | 100 mg/ml (+)-catechin hydrate (5 mg/ml) |
| 5700 μl | 50 mM succinate buffer pH 5.0 |

No horse radish peroxidase control

| | |
|---|---|
| 300 μl | 100 mg/ml (+)-catechin hydrate (5 mg/ml) |
| 600 μl | 3% aqueous hydrogen peroxide (0.3%) |
| 5100 μl | 50 mM succinate buffer pH 5.0 |

No hydrogen peroxide control

| | |
|---|---|
| 300 μl | 100 mg/ml (+)-catechin hydrate (5 mg/ml) |
| 120 μl | 500 U/ml horse radish peroxidase (10 U/ml) |
| 5580 μl | 50 mM succinate buffer pH 5.0 |

Buffer only control

| | |
|---|---|
| 6000 μl | 50 mM succinate buffer pH 5.0 |

The hair switches were then rinsed in distilled water, washed with shampoo, rinsed again as previously and dried using a hair dryer for approximately one minute. Colour generation following treatment was then evaluated using the Minolta model CM508d spectrophotometer. Replicate L*a*b* readings were determined and ΔE values calculated therefrom according to the equation below:

ΔE=√(L*_{B}−L*_{D})²+(a*_{B}−a*_{D})²+(b*_{B}−b*_{D})²

Where B=background/baseline and D=dyed;
L*=lightness (where 0=black and 100=diffuse white);
a*=green/red (negative values indicate green and positive values indicate red); and
b*=blue/yellow (negative values indicate blue and positive values indicate yellow)

Results:

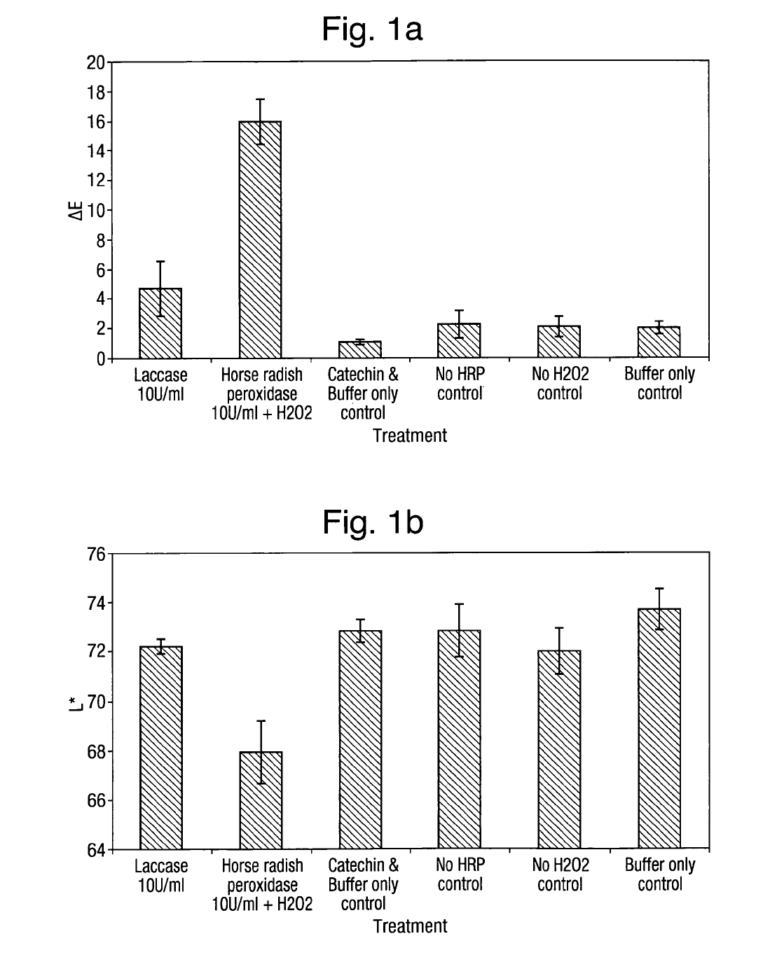

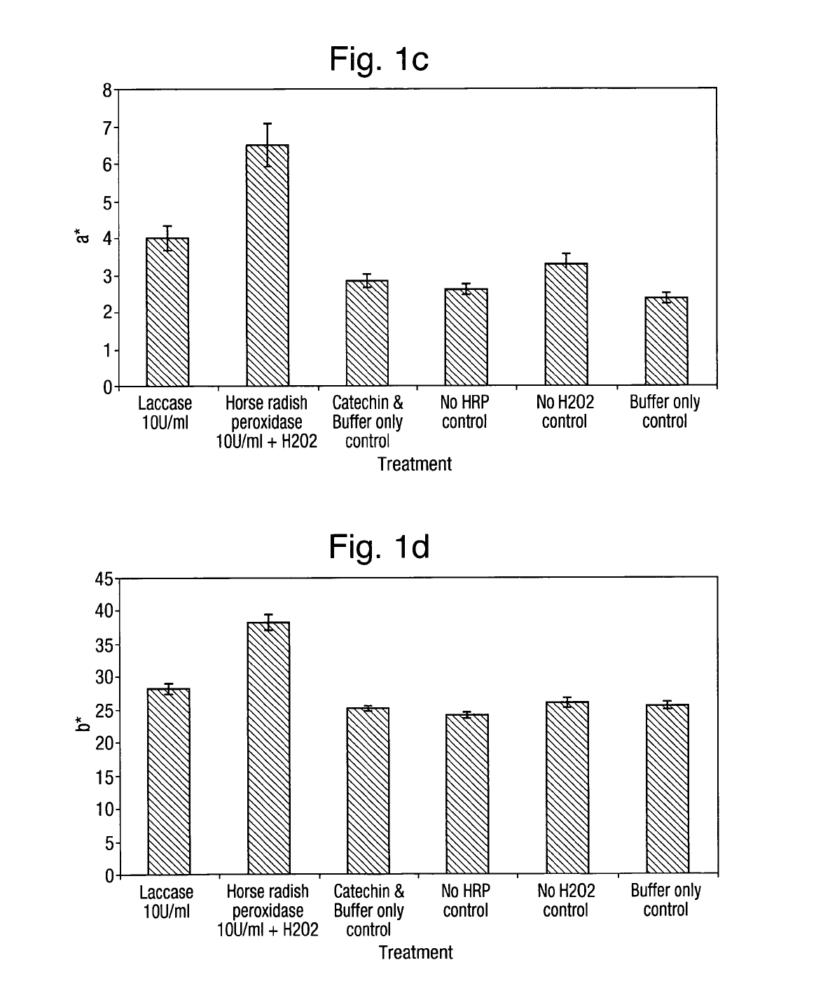

FIGS. 1a, 1b, 1c and 1d show respectively the results for the ΔE and L*, a* and b* for each of the abovementioned reactions on the basis of six repeat measurements at different regions of each hair switch

Conclusion:

Horse radish peroxidase in combination with hydrogen peroxide is more effective than laccase alone for generating colour on hair switches when (+)-catechin hydrate is used as a substrate. In particular, an increase in ΔE was observed using horse radish peroxidase and hydrogen peroxide compared to laccase. In terms of the L*a*b* colour space dimensions, a decrease in L* (lightness) and an increase (more red) in a* (green/red) and an increase (more yellow) in b* (blue/yellow) was observed when using horse radish peroxidase and hydrogen peroxide compared to laccase.

### Example 2

### A Comparison of Cumulative Colour Uptake Over Dyeing Cycles, and Colour Stability after Shampooing Between (+)-Catechin Hydrate and Three Phenylpropenoids Used for Enzymatic Hair Colouring

Materials:

Caffeic acid (3,4-dihydroxycinnamic acid) was obtained from Sigma (UK)

Ferulic acid was obtained from Sigma (UK)

Isoeugenol was obtained from Sigma (UK)

Soybean peroxidase was obtained from Bio-Research Products Incorporated

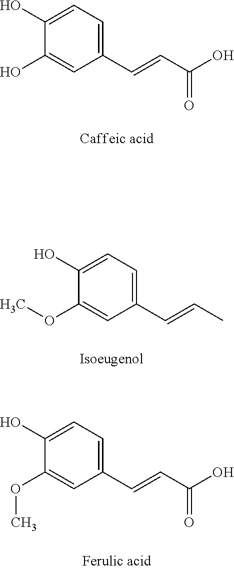

General method:

The general reaction conditions were (final concentrations in brackets):

Oxidation dye precursor (50 mM) (see table below for actual amounts used)

1 ml 3% hydrogen peroxide (0.3%)

50 μl 1 mg/ml or 1840 U/mg soybean peroxidase (5 ppm or 9.4 U/ml)

Balance to 10 ml of 100 mM NaH₂PO4/Na₂HPO4 buffer pH 6.2

which for each oxidation dye precursor meant:

| | | Amount used per 10 ml (g) |
|---|---|---|
| Oxidation dye precursor | % weight | (adjusted for purity) |
| (+)-Catechin | 1.45 | 0.1481 |
| Caffeic acid | 0.90 | 0.0919 |
| Ferulic acid | 0.97 | 0.0981 |
| Isoeugenol | 0.82 | 0.0838 |

Cumulative colour uptake method:

One ˜0.5 g Natural White hair switches were allocated to each dye treatment and five replicate baseline L*a*b* colour measurements per switch taken using a Minolta model CM508d spectrophotometer.

Each of the oxidation dye precursors was weighed out into duplicate glass vials. 2 ml of ethanol (98%) was added to each vial except to isoeugenol where 3 ml was added to maintain solubility. 1 ml of 3% aqueous hydrogen peroxide was then added to each vial and the pH 6.2 phosphate buffer added to make the total volume up to 9.950 ml in each vial.

A switch of hair was placed into each vial ensuring full coverage of the switch by agitation and incubated for 10 minutes. Thereafter 50 μl of a 1 mg/ml soybean peroxidase solution was added to each vial and the switch and dye solution again thoroughly agitated to ensure full coverage of the switch. The switches were incubated for a further 10 minutes and then rinsed in warm running tap water until the water ran clear. The switches were then combed and dried with a hair dryer. Colour measurements were then taken in quintuplicate per hair switch using the Minolta model CM508d spectrophotometer. The above process was repeated five times and cumulative colour build up measured per treatment.

The switches were then subjected to five cycles of washing with shampoo, drying and L*a*b* colour measurements using a Minolta model CM508d spectrophotometer. The switches were washed thoroughly for two minutes by adding 100 μl shampoo per switch, rubbed and combed in shampoo for one minute, and rinsed thoroughly in warm running water. The switches were then dried with a hair dryer.

Results:

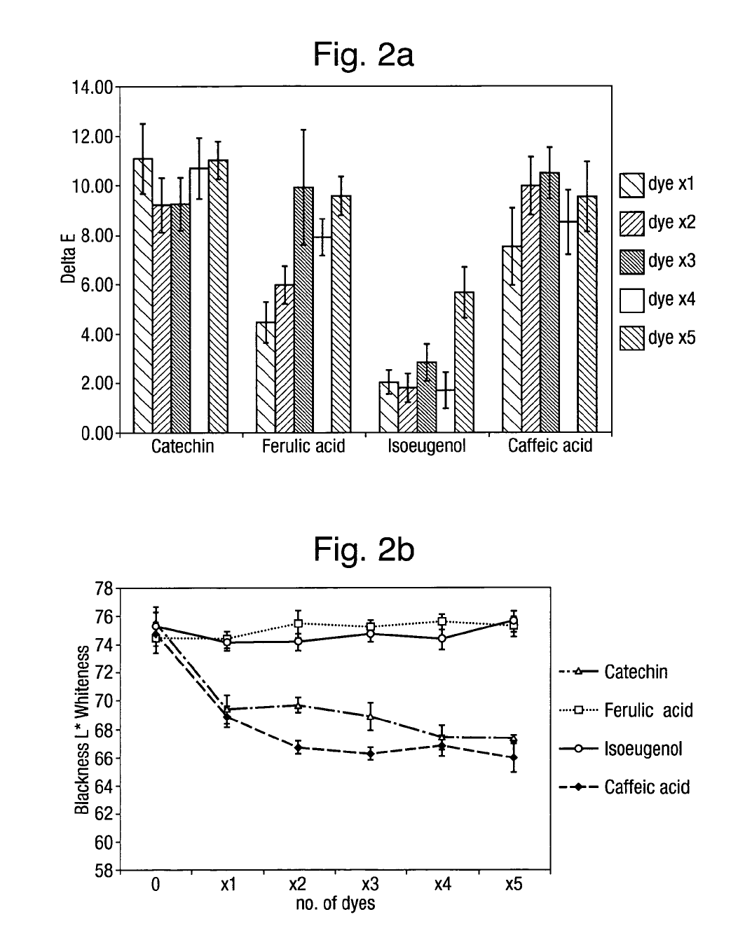

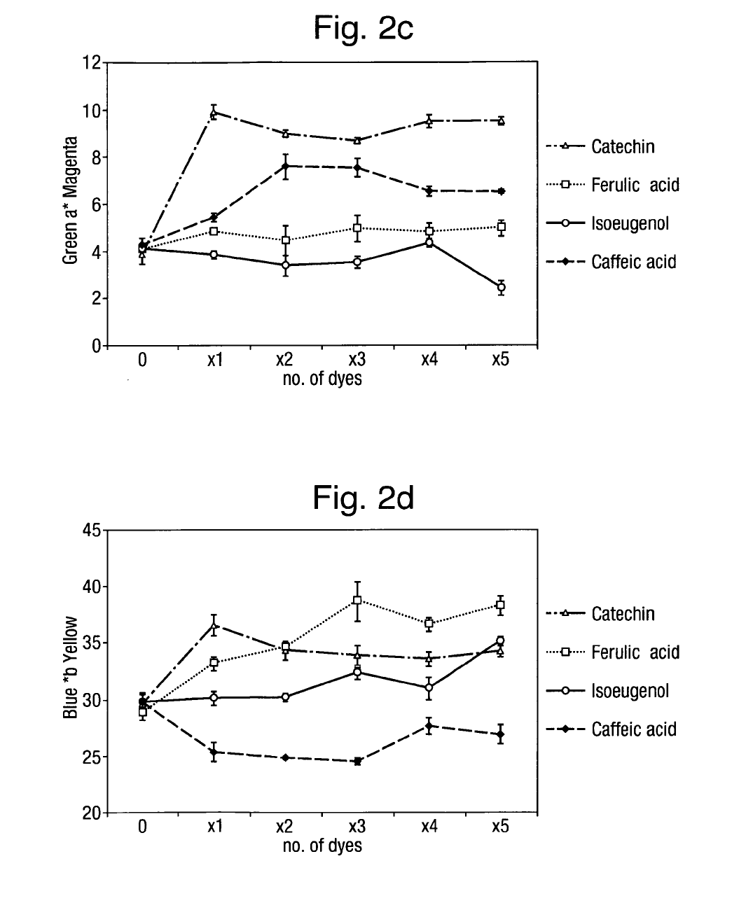

FIGS. 2a, 2b, 2c and 2d show respectively the results for the ΔE and L*, a* and b* for cumulative colour uptake on the basis of five repeat measurements for each hair switch. “0” represents the L*, a* and b* values for untreated natural white hair.

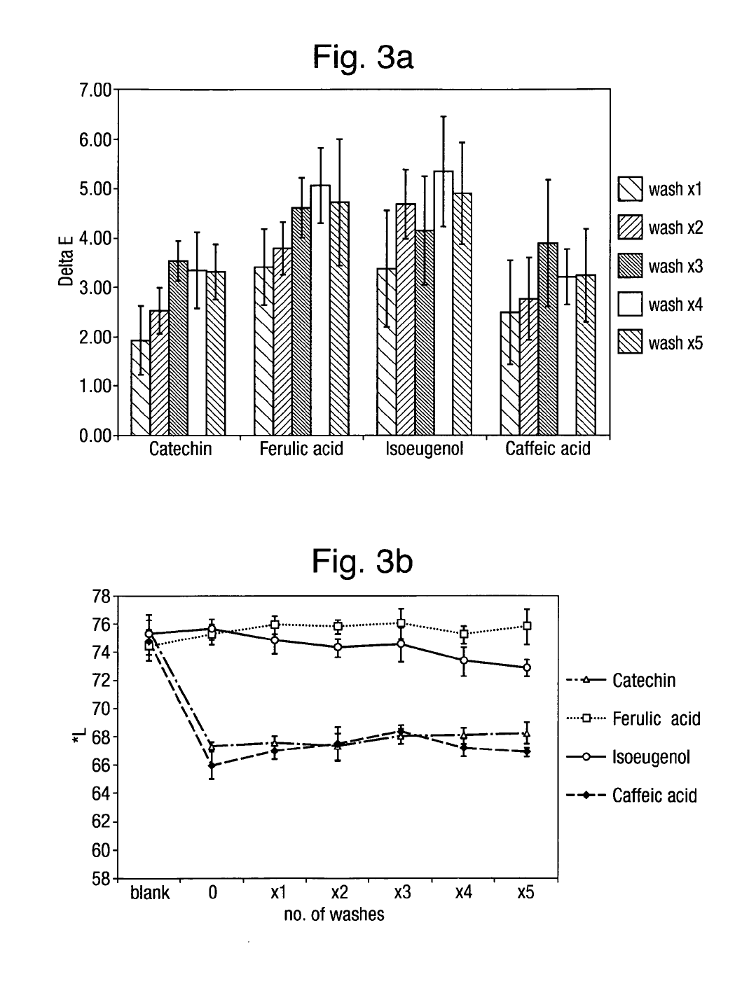

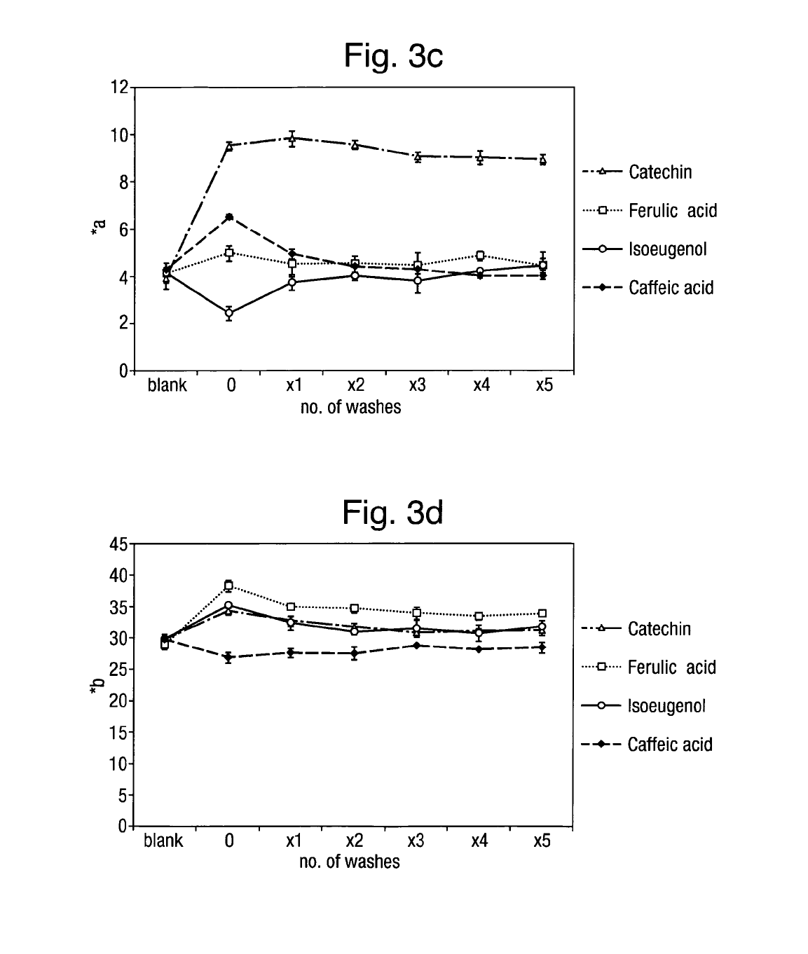

FIGS. 3a, 3b, 3c and 3d show respectively the results for the ΔE and L*, a* and b* for cumulative wash out on the basis of five repeat measurements for each hair switch wherein the ΔE values take the ×5 cumulative colour uptake as the baseline and “0” represents the L*, a* and b* values after ×5 cumulative colour uptake.

Conclusion:

(+)-Catechin hydrate (a flavonoid) shows after a single cycle of colour treatment the greatest colour shift to the red end of the spectrum (a* increases). After five cycles of colour treatment, the (+)-catechin hydrate still shows the greatest colour shift to the red end of the spectrum. This shift to the red end of the spectrum is particularly desired in a hair dye as a good red dye is considered a gold standard in the hair colour industry. Furthermore the shift to the red end of the colour spectrum with (+)-catechin hydrate appears to be colour fast as shown in FIG. 3c.

1. A method of colouring hair fibres, the method comprising the step of applying a hair colour composition, the hair colour composition comprising:
(a) (+)-Catechin, (−)-catechin, (+)-epicatechin, (−)-epicatechin or mixtures thereof;
(b) A hydrogen peroxide generator or hydrogen peroxide; and
(c) A peroxidase and;
wherein the composition has a pH of 4.5 to 7.0, preferably less than or equal to 6.0.

2. A method of colouring hair fibres according to claim 1, wherein the peroxidase is a non-animal haem peroxidase from class II (fungi) or class III (plants and algae).

3. A method of colouring hair fibres according to claim 2, wherein the peroxidase is obtained from the group consisting of Arabidopsis thaliana, horse radish, barley, peanut soybean, tobacco, and turnip (plants), Chlorophyta spirogyra (green algae), Arthromyces ramosus and Corprinus cinereus (fungi).

4. A method of colouring hair fibres according to claim 1, wherein the hair colour composition comprises 0.01-10, preferably 0.1-5% w/w (+)-catechin, (−)-catechin (+)-epicatechin, (−)-epicatechin or mixtures thereof.

5. A method of colouring hair fibres according to claim 1, wherein the hair colour composition comprises 0.0001-3 preferably 0.001-1, most preferably 0.01-1% w/w hydrogen peroxide.

6. A method of colouring hair fibres according to claim 1, wherein the hair colour composition comprises 0.0001-5, preferably 0.001-1% w/w peroxidase.

7. A method of colouring hair fibres according to claim 1, wherein the hydrogen peroxide generator comprises a hydrogen peroxide generating oxidase, a substrate and oxygen.

8. A method of colouring hair fibres according to claim 1, wherein the hydrogen peroxide generating oxidase is selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, α-hydroxy acids, D-glucose, oxalic acid, amino acid and L-galactono-1,4-lactone.

9. A method of colouring hair fibres according to claim 1 wherein the hydrogen peroxide generator is selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, and L-galactono-1,4-lactone with L-galactonolactone oxidase, amino acid oxidase with amino acids, all in the presence of oxygen.

10. A method of colouring hair fibres according to claim 1, wherein the hair colour composition comprises 0.0001-5, preferably 0.001-1% w/w hydrogen peroxide generating oxidase.

11. A method of colouring hair fibres according to claim 1 wherein the hair colour composition comprises 0.01-10 preferably 0.1-5% w/w substrate.

12. A method of colouring hair fibres according to claim 1 additionally comprising a metal ion suitable for coordinating to (+)-catechin, (−)-catechin, (+)-epicatechin, (−)-epicatechin or mixtures thereof, or the product of the reaction of (+)-catechin, (−)-catechin, (+)-epicatechin, (−)-epicatechin or mixtures thereof in the presence of the peroxidase and hydrogen peroxide.

13. A method of colouring hair fibres according to claim 1, wherein the metal ion is selected from the group consisting of iron (II), iron (III), copper (I), copper (II), copper (III) and aluminium (III).

14. A method of colouring hair fibres according to claim 1, wherein the hair colour composition comprises 0.0001-2, preferably 0.001-0.1% w/w metal ion.
